SEQUENCE LISTING (29 sequences)

SEQ ID NO: 1 - protein, 6 aa, Artificial
  CDR1 from light chain of anti-CD160 antibody
QSISNH

SEQ ID NO: 2 - protein, 9 aa, Artificial
  CDR3 from light chain of anti-CD160 antibody
QQSNSWPLT

SEQ ID NO: 3 - protein, 8 aa, Artificial
  CDR1 from heavy chain of anti-CD160 antibody
GYTFTDYW

SEQ ID NO: 4 - protein, 8 aa, Artificial
  CDR2 from heavy chain of anti-CD160 antibody
IYPGDDDA

SEQ ID NO: 5 - protein, 14 aa, Artificial
  CDR3 from heavy chain of anti-CD160 antibody
ARRGIAAVVGGFDY

SEQ ID NO: 6 - protein, 121 aa, Mus musculus
QVHLQQSGAELARPGASVKLSCKASGYTFTDYWMQWIKQRPGQGLEWIGSIYPGDDDARYTQKFRGKATLTADKSSSTAYMQLSSLASEDSAVYYCARRGIAAVVGGFDYWGQGTTLTVSS

SEQ ID NO: 7 - DNA, 363 bp, Mus musculus
caggttcacctccagcagtctggggctgagctggcaagacctggggcttcagtgaagttgtcctgcaaggcttctggctacacctttactgactactggatgcagtggataaaacagaggcctggacagggtctggaatggattgggtctatttatcctggagatgatgatgctaggtacactcagaagttcaggggcaaggccacattgactgcagataaatcctccagcacagcctacatgcagctcagcagcttggcatctgaagactctgcggtctattactgtgcaagaaggggtattgctgcggtagtaggcggctttgactactggggccaaggcaccactctcacagtctcctca

SEQ ID NO: 8 - protein, 108 aa, Mus musculus
DIVLTQSPATLSVTPGNSVSLSCRASQSISNHLHWYQQKSHESPRLLIKYASQSISGIPSRFSGSGSGTDFTLSINSVETEDFGMYFCQQSNSWPLTFGAGTKLELKR

SEQ ID NO: 9 - DNA, 324 bp, Mus musculus
gatattgtgctaactcagtctccagccaccctgtctgtgactccaggaaatagcgtcagtctttcctgcagggccagccaaagtattagcaaccacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggcatgtatttctgtcaacagagtaacagctggccgctcacgttcggtgctgggaccaagctggagctaaaacgg

SEQ ID NO: 10 - protein, 114 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPC

SEQ ID NO: 11 - DNA, 345 bp, homo sapiens
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccatgctaa

SEQ ID NO: 12 - protein, 330 aa, homo sapiens [sequence omitted]

SEQ ID NO: 13 - DNA, 993 bp, homo sapiens
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 14 - protein, 327 aa, homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - DNA, 984 bp, homo sapiens
gctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaataa

SEQ ID NO: 16 - protein, 107 aa, homo sapiens [sequence omitted]

SEQ ID NO: 17 - DNA, 324 bp, homo sapiens
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaa

SEQ ID NO: 18 - protein, 130 aa, mus musculus
MDMRVPAQLLGLLLLWLPGARCDIVLTQSPATLSVTPGNSVSLSCRASQSISNHLHWYQQKSHESPRLLIKYASQSISGIPSRFSGSGSGTDFTLSINSVETEDFGMYFCQQSNSWPLTFGAGTKLELKR

SEQ ID NO: 19 - DNA, 390 bp, mus musculus
atggacatgcgtgtgcccgctcaactcctgggcctgctgctgctctggctcccaggtgcgcgctgtgatattgtgctaactcagtctccagccaccctgtctgtgactccaggaaatagcgtcagtctttcctgcagggccagccaaagtattagcaaccacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggcatgtatttctgtcaacagagtaacagctggccgctcacgttcggtgctgggaccaagctggagctaaaacgg

SEQ ID NO: 20 - protein, 140 aa, mus musculus
MEFGLSWLFLVAILKGVQCQVHLQQSGAELARPGASVKLSCKASGYTFTDYWMQWIKQRPGQGLEWIGSIYPGDDDARYTQKFRGKATLTADKSSSTAYMQLSSLASEDSAVYYCARRGIAAVVGGFDYWGQGTTLTVSS

SEQ ID NO: 21 - DNA, 420 bp, mus musculus
atggagttcggcctgagctggctgttcctggtggctattcttaagggtgtccagtgtcaggttcacctccagcagtctggggctgagctggcaagacctggggcttcagtgaagttgtcctgcaaggcttctggctacacctttactgactactggatgcagtggataaaacagaggcctggacagggtctggaatggattgggtctatttatcctggagatgatgatgctaggtacactcagaagttcaggggcaaggccacattgactgcagataaatcctccagcacagcctacatgcagctcagcagcttggcatctgaagactctgcggtctattactgtgcaagaaggggtattgctgcggtagtaggcggctttgactactggggccaaggcaccactctcacagtctcctca

SEQ ID NO: 22 - protein, 107 aa, homo sapiens
EIVLTQSPATLSLSPGERATLSCRASQSINNYLAWYQQKDGQAPRLLIYDASNRATGIPTRFSGSGSGTDFTLTINSLEPEDFAVYYCQQRNNWPLTFGGGTKVEIK

SEQ ID NO: 23 - protein, 114 aa, homo sapiens
QVQLLESGAELVKPGASVKLSCKASGYTFTSYWMHWVKQRPGRGLEWIGMIDPNSGGTKYNEKFKSKATLTVDKPSNTAYMQLSSLTSEDSAVYYCTRRDMDYWGAGTTVTVSS

SEQ ID NO: 24 - protein, 114 aa, homo sapiens
  misc_feature (3)..(3): Xaa can be any naturally occurring amino acid
EVXLQZSGTVLARPGASVRMSCKASGYSFTSYWLHWIKQRPGQGLEWIGGIYPGNRDTRYTQRFKDKAKLTAVTSANTAYMELSSLTNEDSAVYYCSIIYFDYADFIMDYWGQG

SEQ ID NO: 25 - protein, 108 aa, artificial
  humanized variable light chain anti-CD160 antibody
DIVLTQSPATLSVTPGXXXSLSCRASQSISNHLHWYQQKSXESPRLLIKYASXSISGIPSRFSGSGSGTDFTLSINSVEXEDFXMYFCQQSNSWPLTFGXGTKLELKR

SEQ ID NO: 26 - protein, 121 aa, artificial
  humanized variable heavy chain anti-CD160 antibody
QVXLQQSGAELARPGASVKLSCKASGYTFTDYWMXWIKQRPGQGLEWIGSIYPGDDDARYTQKFRGKATLTADKSSSTAYMQLSSLXSEDSAVYYCARRGIAAVVGGFDYWGQGTTLTVSS

SEQ ID NO: 27 - protein, 181 aa, homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQAL

SEQ ID NO: 28 - protein, 129 aa, Mus musculus
MDMRVPAQLLGLLLLWLPGARCDIVLTQSPATLSVTPGNSVSLSCRASQSISNHLHWYQQKSHESPRLLIKYASQSISGIPSRFSGSGSGTDFTLSINSVETEDFGMYFCQQSNSWPLTFGAGTKLELK

SEQ ID NO: 29 - DNA, 387 bp, Mus musculus
atggacatgcgtgtgcccgctcaactcctgggcctgctgctgctctggctcccaggtgcgcgctgtgatattgtgctaactcagtctccagccaccctgtctgtgactccaggaaatagcgtcagtctttcctgcagggccagccaaagtattagcaaccacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggcatgtatttctgtcaacagagtaacagctggccgctcacgttcggtgctgggaccaagctggagctaaaa

(19) United States
(12) Patent Application Publication
(10) Pub. No.: US 2011/0280866 A1
(43) Pub. Date: Nov. 17, 2011

(54) ANTI-CD 160 MONOCLONAL ANTIBODIES AND USES THEREOF

(75) Inventors: Denis Didierlaurent, Clichy Sous Bois (FR); Olivier Chose, Paris (FR); Jean Kadouche, Paris (FR)

(73) Assignees: INSTITUT NATIONAL DE LA SANTE ET DE LA RECHERCHE MEDICALE (INSERM); MONOCLONAL ANTIBODIES THERAPECTICS

(21) Appl. No.: 13/145,509

(22) PCT Filed: Jan. 21, 2010
(86) PCT No.: PCT/EP10/50694

§ 371 Date: Jul. 20, 2011

(30) Foreign Application Priority Data
Jan. 21, 2009 (EP) 09305057.3

(51) Int. Cl. A61K 39/395; A61P 19/02; A61P 35/00; C07K 16/28

(52) U.S. Cl. 424/130.1; 424/133.1; 530/387.3; 530/389.1

(57) ABSTRACT

The present invention concerns an antibody or fragment thereof, capable of binding to CD 160, said antibody comprising a) a light chain comprising three light chain complementary regions (CDRs) having the following amino acid sequences: (i) the light chain CDR1: QSISNH (SEQ ID NO: 1), (ii) the light chain CDR2: YAS, (iii) the light chain CDR3: QQSNSWPLT (SEQ ID NO: 2), and a light chain framework sequence from an immunoglobulin light chain; and b) a heavy chain comprising three heavy chain complementary regions (CDRs) having the following amino acid sequences: (i) the heavy chain CDR1: GYTFTDYW (SEQ ID NO: 3), (ii) the heavy chain CDR2: IYPGDDDA (SEQ ID NO: 4); (iii) the heavy chain CDR3: ARRGIAAVVGGFDY (SEQ ID NO: 5); and a heavy chain framework sequence from an immunoglobulin heavy chain; a pharmaceutical comprising said antibody and the use of said antibody for preparation of a medicament for treating and/or preventing a pathology associated with endothelial cells proliferation engaged in an angiogenesis process.

### FIELD OF THE INVENTION

The present invention provides novel anti-CD160 monoclonal antibodies, in particular mouse-human chimeric anti-CD160 antibodies. Anti-CD160 antibodies, as well as pharmaceutical compositions containing them, are useful for inhibiting angiogenesis.

### BACKGROUND OF THE INVENTION

NK cells major effector functions are characterized by cytolytic activity and the production of cytokines and chemokines directed against susceptible target cells. NK cell discriminate between healthy and abnormal tumor cells or virally infected cells through specific engagement of various activating receptors. Most activating NK cell receptors can be divided in three groups according their signalling mediated associated molecules, but a unique distinct activating NK cell receptor has been identified, CD160, which is an MHC class I-dependent immunoglobulin (Ig)-like molecule.

CD160 is expressed by human and mouse circulating cytotoxic lymphocytes, as NK CD56^{dim+}CD16⁺, most TCRγδT cells, and cytotoxic effector TCRαβCD8T cells, and also in almost all intestinal intraepithelial lymphocytes (iIELs) (MAIZA et al., J. Exp. Med., vol. 178, p: 1124-6, 1993; ANUMANTHAN et al., J. Immunol., vol. 161, p: 2780-90, 1998).

Finally, CD160 exhibits the following unique characteristics when compared to the other activating receptors described to date: 1) CD160 is encoded by a gene on chromosome 1 outside the NK gene complex, 2) it is a multimeric glycosyl phosphatidyl inositol (GPI)-anchored molecule, 3) its cell surface expression is down-modulated after activation.

Angiogenesis is a fundamental process by means of which new capillaries from the pre-existing blood vessels are formed. This process is essential in many normal physiological phenomena such as reproduction, differentiation, cicatrisation, and organ regeneration.

Angiogenesis is under strict control in these normal biological phenomena; i.e., it is triggered during a brief period of several days and then completely inhibited. However, many pathologies are linked to invasive, uncontrolled angiogenesis. Arthritis, for example, is a pathology caused by damage caused to cartilage by invasive neovessels. In diabetic retinopathy, the invasion of the retina by neovessels results in the patients going blind; neovascularization of the ocular apparatus is the major cause of blindness and this neovascularization dominates at least twenty diseases of the eye. Lastly, the growth and metastasis of many tumors are directly dependent on angiogenesis. The tumor stimulates the growth of the neovessels for its own use. Furthermore, these neovessels present escape routes by means of which the tumors can reach the blood circulatory system and cause metastases in remote sites such as the liver, lungs or bones.

Recently, CD160 has been identified as an inhibitory signalling receptor for angiogenesis (FONS et al., Blood, vol. 108(8), p: 2608-15, 2006).

WO 03/018048 in the name of ABTECH and INSERM relates to the use of two soluble HLA class I molecules that bind CD160, namely sHLA-G1 and sHLA-B7, to inhibit angiogenesis.

WO 2006/015886 in the name of INSERM relates to the use of an anti-CD160 monoclonal antibody for inhibiting vessel formation and growth that is induced by pro-angiogenic factors such as VEGF or FGF2 on endothelial cells.

### SUMMARY OF THE INVENTION

The present invention relates to an antibody, or fragment thereof, capable of binding to CD160, having a polypeptidic sequence comprising at least 90% homology with the polypeptidic sequence of an antibody comprising:

a) a light chain comprising three light chain complementary regions (CDRs) having the following amino acid sequences:
i) the light chain CDR1: QSISNH (SEQ ID NO:1);
ii) the light chain CDR2: YAS;
iii) the light chain CDR3: QQSNSWPLT (SEQ ID NO: 2); and
a light chain framework sequence from an immunoglobulin light chain; and

b) a heavy chain comprising three heavy chain complementary regions (CDRs) having the following amino acid sequences:
i) the heavy chain CDR1: GYTFTDYW (SEQ ID NO: 3);
ii) the heavy chain CDR2: IYPGDDDA (SEQ ID NO: 4);
iii) the heavy chain CDR3: ARRGIAAVVGGFDY (SEQ ID NO: 5); and
a heavy chain framework sequence from an immunoglobulin heavy chain.

The present invention also relates to a pharmaceutical composition comprising at least one of such anti-CD160 antibody or fragment thereof, and a pharmaceutically acceptable carrier.

Additionally, the present invention relates to a method for inhibiting angiogenesis comprising providing to a patient in need thereof such a pharmaceutical composition.

Finally, the present invention relates to the use of at least one of such anti-CD160 antibody or fragment thereof for the preparation of a medicament for treating and/or preventing a pathology associated with endothelial cells proliferation engaged in an angiogenesis process.

### BRIEF DESCRIPTION OF THE FIGURES:

FIG. 1: Amino acid sequence (SEQ ID No.6) and nucleotide sequence (SEQ ID No.7) of the variable region of the heavy chain of the mouse CD160 antibody.

FIG. 2: Amino acid sequence (SEQ ID No.8) and nucleotide sequence (SEQ ID No.9) of the variable region of the light chain of the mouse CD160 antibody.

FIG. 3: Amino acid sequence (SEQ ID No.10) and nucleotide sequence (SEQ ID No.11) derived from the constant region of the heavy chain of human gamma 1 immunoglobulins by the insertion of an additional cysteine and of a stop codon.

FIG. 4: Amino acid sequence (SEQ ID No.12) and nucleotide sequence (SEQ ID No.13) of the constant region of the heavy chain of human gamma 1 immunoglobulins. The first AGC codon was modified in GCT in order to create a cloning site (NheI).

FIG. 5: Amino acid sequence (SEQ ID No.14) and nucleotide sequence (SEQ ID No.15) of the constant region of the heavy chain of human gamma 4 immunoglobulins. The first AGC codon was modified in GCT in order to create a cloning site (NheI).

FIG. 6: Amino acid sequence (SEQ ID No.16) and nucleotide sequence (SEQ ID No.17) of the constant region of the light chain of human kappa immunoglobulins.

FIG. 7: a) Amino acid sequence (SEQ ID No.18) and nucleotide sequence (SEQ ID No.19) of a recombinant variable region of the light chain of the mouse CD160 antibody comprising the human VL signal peptide (shared). b) Amino acid sequence (SEQ ID No.28) and nucleotide sequence (SEQ ID No.29) of a recombinant variable region of the light chain of the mouse CD160 antibody comprising the human VL signal peptide (shared).

FIG. 8: Amino acid sequence (SEQ ID No.20) and nucleotide sequence (SEQ ID No.21) of a recombinant variable region of the heavy chain of the mouse CD160 antibody comprising the human VH signal peptide (shared).

FIG. 9: Alignment between amino acid sequence the mouse variable light chain (SEQ ID No.8) and the human immunoglobulin having the maximum of homology (SEQ ID No. 22; Accession number AAZ09098). The CDR regions are identified (shared).

FIG. 10: Alignment between amino acid sequence the mouse variable heavy chain (SEQ ID No.6) and one of the human immunoglobulin having the maximum of homology (SEQ ID No. 23; Accession number 3FCTB).The CDR regions are identified (shared).

FIG. 11: Alignment between amino acid sequence the mouse variable heavy chain (SEQ ID No.6) and one of the human immunoglobulin having the maximum of homology (SEQ ID No. 24; Accession number AAG00910).The CDR regions are identified (shared).

### DETAILED DESCRIPTION:

In a first aspect, the present invention concerns an antibody, or fragment thereof, capable of binding to CD160, said antibody having a polypeptidic sequence comprising at least 90% homology, preferably 95%, and more preferably 98% with the polypeptidic sequence of an antibody comprising:

a) a light chain comprising three light chain complementary regions (CDRs) having the following amino acid sequences:

i) the light chain CDR1: QSISNH (SEQ ID NO:1);
ii) the light chain CDR2: YAS;
iii) the light chain CDR3: QQSNSWPLT (SEQ ID NO: 2); and
a light chain framework sequence from an immunoglobulin light chain; and b) a heavy chain comprising three heavy chain complementary regions (CDRs) having the following amino acid sequences:

i) the heavy chain CDR1: GYTFTDYW (SEQ ID NO: 3);
ii) the heavy chain CDR2: IYPGDDDA (SEQ ID NO: 4);
iii) the heavy chain CDR3: ARRGIAAVVGGFDY (SEQ ID NO: 5); and
a heavy chain framework sequence from an immunoglobulin heavy chain.

An antibody is an immunoglobulin molecule corresponding to a tetramer comprising four polypeptide chains, two identical heavy (H) chains (about 50-70 kDa when full length) and two identical light (L) chains (about 25 kDa when full length) inter-connected by disulfide bonds. Light chains are classified as kappa and lambda. Heavy chains are classified as gamma, mu, alpha, delta, or epsilon, and define the antibody's isotype as IgG, IgM, IgA, IgD, and IgE, respectively. Each heavy chain is comprised of a N-term heavy chain variable region (abbreviated herein as HCVR) and a heavy chain constant region. The heavy chain constant region is comprised of three domains (CH1, CH2, and CH3) for IgG, IgD, and IgA; and 4 domains (CH1, CH2, CH3, andCH4) for IgM and IgE. Each light chain is comprised of a N-term light chain variable region (abbreviated herein as LCVR) and a light chain constant region. The light chain constant region is comprised of one domain, CL. The HCVR and LCVR regions can be further subdivided into regions of hypervariability, termed complementarity determining regions (CDRs), interspersed with regions that are more conserved, termed framework regions (FR). Each HCVR and LCVR is composed of three CDRs and four FRs, arranged from amino-terminus to carboxy-terminus in the following order: FR1, CDR1, FR2, CDR2, FR3, CDR3, FR4. The assignment of amino acids to each domain is in accordance with well-known conventions (KABAT, “Sequences of Proteins of Immunological Interest”, National Institutes of Health, Bethesda, Md., 1987 and 1991; CHOTHIA et al., J. Mol. Biol., vol. 196, p: 901-17, 1987; CHOTHIA et al., Nature, vol. 342, p: 878-83, 1989). The functional ability of the antibody to bind a particular antigen depends on the variable regions of each light/heavy chain pair, and is largely determined by the CDRs.

The term “antibody”, as used herein, refers to a monoclonal antibody per se. A monoclonal antibody can be a human antibody, chimeric antibody and/or humanized antibody.

When used herein, when a “polypeptidic sequence A comprises at least X % homology with the polypeptidic sequence of B”, it means that the amino acid sequence of the polypeptide A has at least X % percentage of identity with the amino acid sequence of B. As used herein, “percentage of identity” between two amino acid sequences denotes the percentage of amino acids residues that are identical between the two sequences to be compared, obtained after the best alignment (optimum alignment), this percentage being purely statistical and the differences between the two sequences being distributed randomly and along their entire length. Sequence comparisons between two amino acid sequences can be performed for example with the BLAST program available on the website http://www.ncbi.nlm.nih.gov/gorf/b12.html, the parameters used being those given by default (in particular for the parameters “open gap penalty”:5 and “extension gap penalty”:2, the matrix selected being for example the “BLOSUM 62” matrix as suggested by the program, the percentage identity between the two sequences to be compared being calculated directly by the program).

The term “fragments” as used herein refers to antibody fragments that bind to a CD160. For example, antibody fragments capable of binding to CD160 include Fab (e.g., by papain digestion), Fab' (e.g., by pepsin digestion and partial reduction) and F(ab')2 (e.g., by pepsin digestion), facb (e.g., by plasmin digestion), pFc' (e.g., by pepsin or plasmin digestion), Fd (e.g., by pepsin digestion, partial reduction and reaggregation), Fv or scFv (e.g., by molecular biology techniques) fragments, are encompassed by the invention.

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab')2 heavy chain portion can be designed to include DNA sequences encoding the CH₁ domain and/or hinge region of the heavy chain. The various portions of antibodies can be joined together chemically by conventional techniques, or can be prepared as a contiguous protein using genetic engineering techniques.

The expression “capable of binding to CD160” refers to a K_{D} of less than 10⁻⁷ M, preferably from less than 10⁻⁸ M, and more preferably less than 10⁻⁹ M for CD 160.

Preferably, but not necessarily, the antibodies useful in the invention are produced recombinantly, as manipulation of the typically murine or other non-human antibodies with the appropriate specificity is required in order to convert them to humanized form. Antibodies may or may not be glycosylated, though glycosylated antibodies are preferred. Antibodies are properly cross-linked via disulfide bonds, as is well-known.

As is well understood in the art, monoclonal antibodies can readily be generated with appropriate specificity by standard techniques of immunization of mammals forming produces it. These nucleotide sequences can then be manipulated to provide them in humanized form.

By “chimeric antibody” is meant an antibody that is composed of variables regions from a murine immunoglobulin and of constant regions of a human immunoglobulin. This alteration consists simply of substituting the constant region of a human antibody for the murine constant region, thus resulting in a human/murine chimera which may have sufficiently low immunogenicity to be acceptable for pharmaceutical use.

A number of methods for producing such chimeric antibodies have yet been reported, thus forming part of the general knowledge of the skilled artisan (See, e.g., U.S. Pat. No. 5,225,539).

In a preferred embodiment, said antibody is a chimeric antibody and the light and heavy chain framework sequences are from mouse immunoglobulin light and heavy chains respectively.

Preferably, the antibody of the present invention comprises:

a) a light chain comprising three light chain complementary regions (CDRs) having amino acid sequences having at least 90%, preferably 95%, and more preferably 98% homology with the following amino acid sequences i) to iii):
i) the light chain CDR1: QSISNH (SEQ ID NO:1);
ii) the light chain CDR2: YAS;
iii) the light chain CDR3: QQSNSWPLT (SEQ ID NO: 2); and
a light chain framework sequence from an immunoglobulin light chain; and

b) a heavy chain comprising three heavy chain complementary regions (CDRs) having amino acid sequences having at least 90% preferably 95%, and more preferably 98% homology with the following amino acid sequences i) to iii):
i) the heavy chain CDR1: GYTFTDYW (SEQ ID NO: 3);
ii) the heavy chain CDR2: IYPGDDDA (SEQ ID NO: 4);
iii) the heavy chain CDR3: ARRGIAAVVGGFDY (SEQ ID NO: 5); and
a heavy chain framework sequence from an immunoglobulin heavy chain.

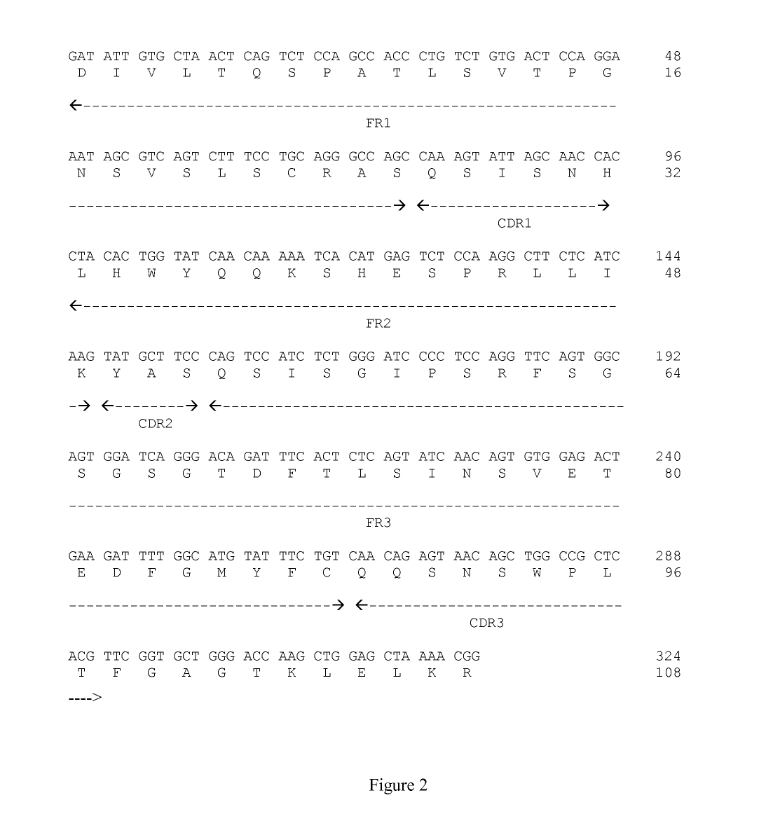

In a particular embodiment of this preferred embodiment, said antibody comprises the light chain variable region (LCVR) with the amino acid sequence SEQ ID NO: 8 shown in FIG. 2. Preferably, said antibody comprises the light chain variable region (LCVR) encoded by the nucleic acid sequence SEQ ID NO:9 shown in FIG. 2.

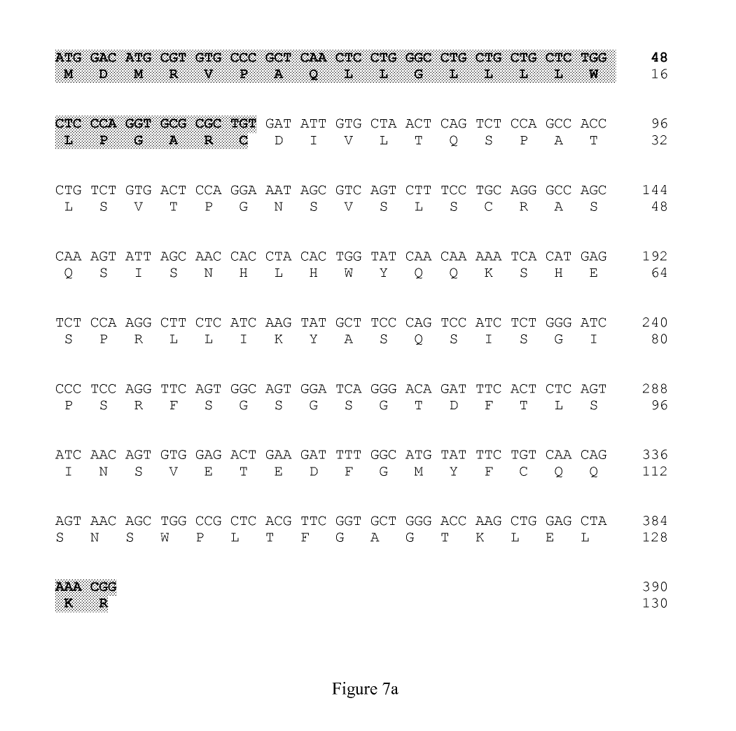

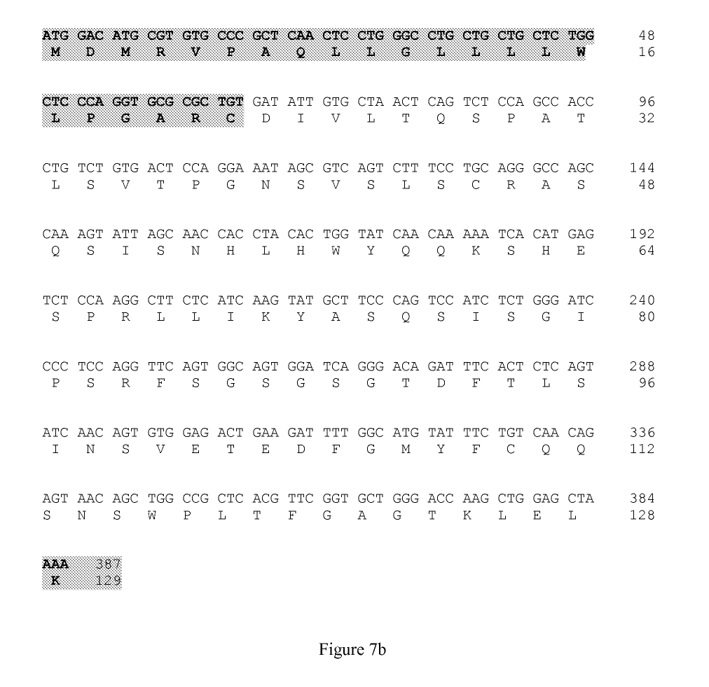

Advantageously, said antibody comprises the light chain variable region (LCVR) with the amino acid sequence SEQ ID NO: 18 shown in FIG. 7a or the amino acid sequence SEQ ID NO:28 shown in FIG. 7b. Preferably, said antibody comprises the light chain variable region (LCVR) encoded by the nucleic acid sequence SEQ ID NO: 19 shown in FIG. 7a or the nucleic acid sequence SEQ ID NO:29 shown in FIG. 7b.

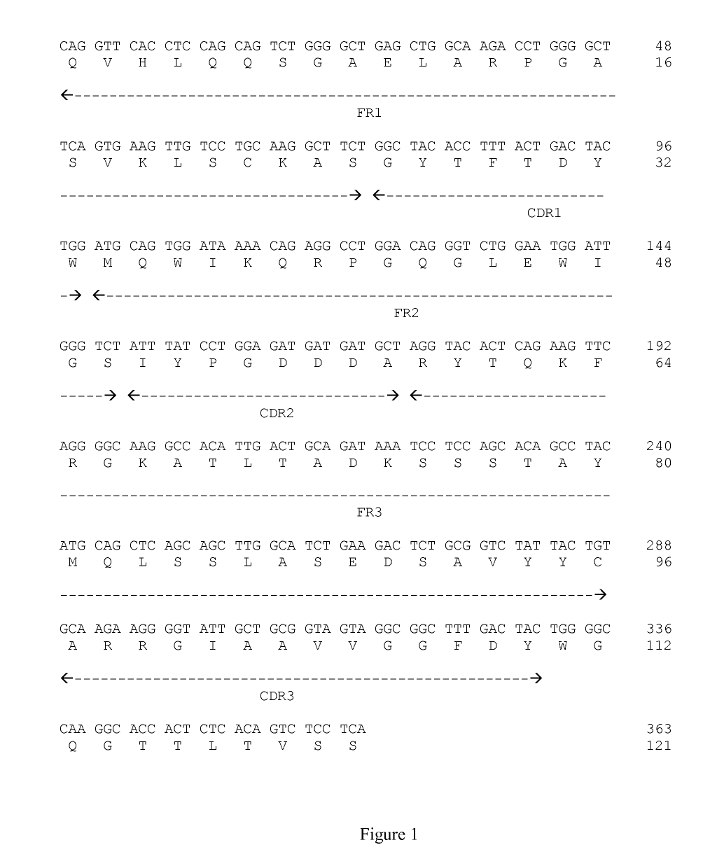

In another particular embodiment of this preferred embodiment, said antibody comprises the heavy chain variable region (HCVR) with the amino acid sequence SEQ ID NO:6 shown in FIG. 1. Preferably, said antibody comprises the heavy chain variable region (HCVR) encoded by the nucleic acid sequence SEQ ID NO:7 shown in FIG. 1.

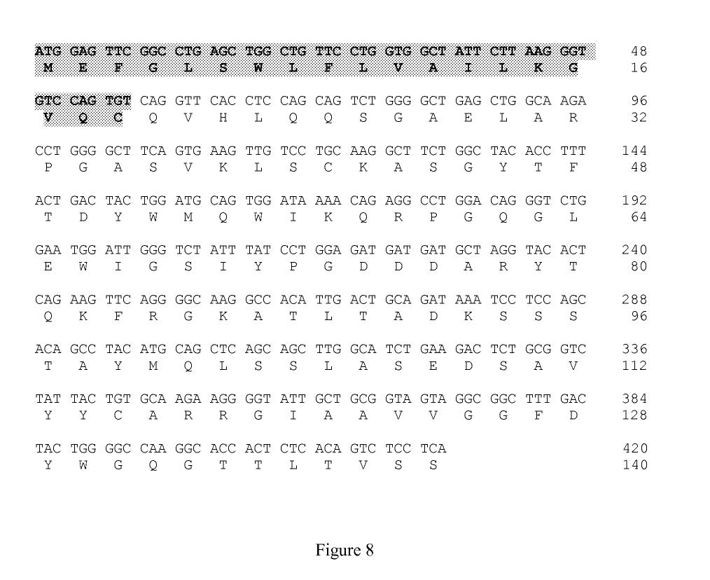

Advantageously, said antibody comprises the heavy chain variable region (HCVR) with the amino acid sequence SEQ ID NO: 20 shown in FIG. 8. Preferably, said antibody comprises the heavy chain variable region (HCVR) encoded by the nucleic acid sequence SEQ ID NO:21 shown in FIG. 8.

In still another particular embodiment of this preferred embodiment, said antibody further comprises the constant regions from human light and heavy chains.

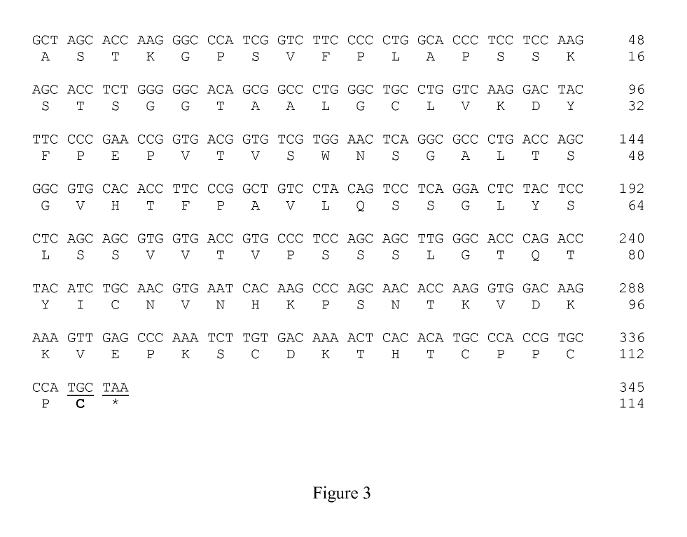

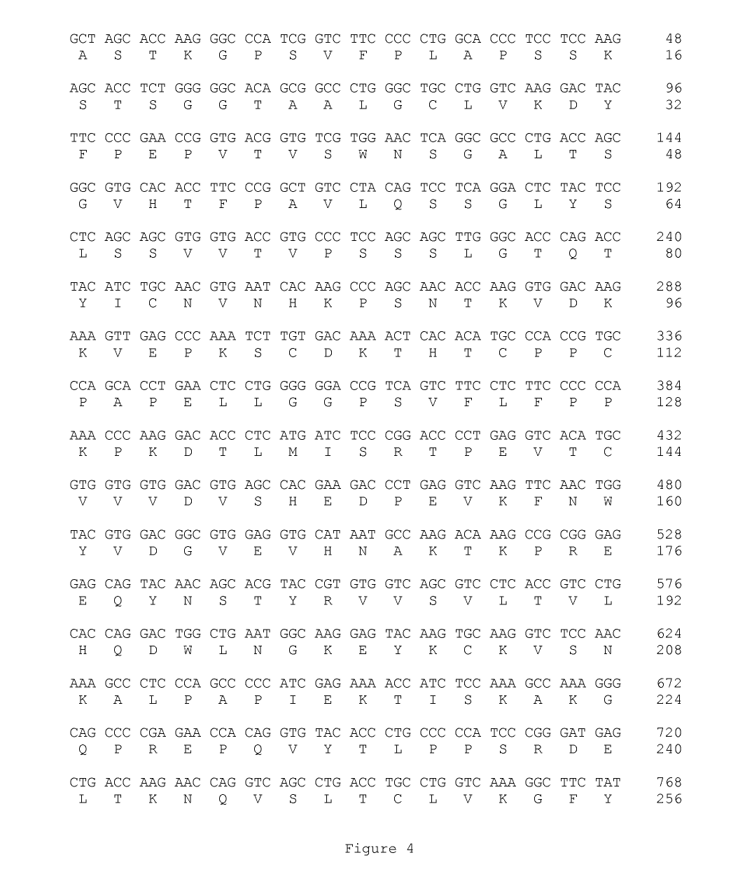

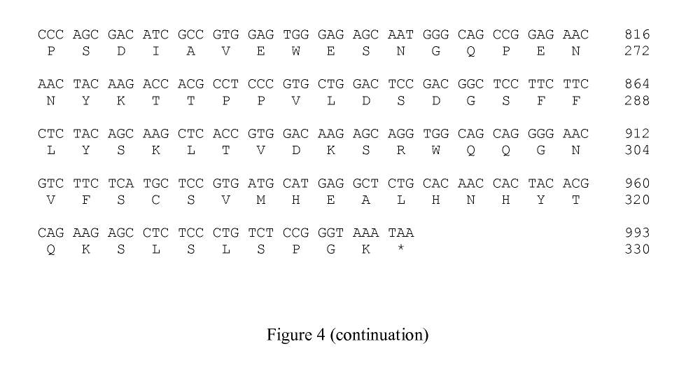

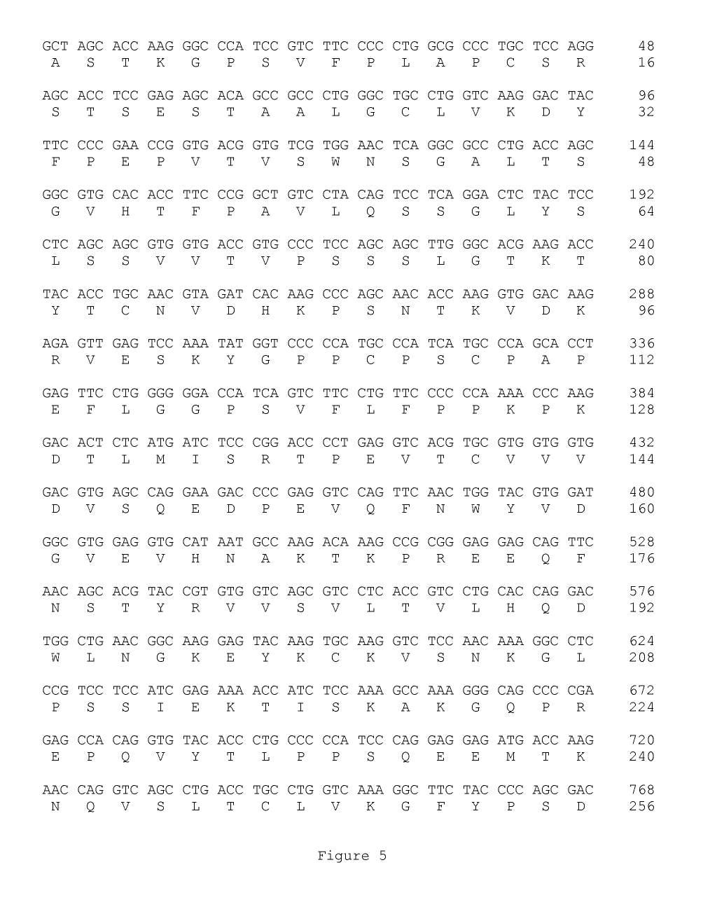

Advantageously, said antibody comprises a constant region from a human heavy chain selected in the group comprising:
the recombinant constant region of heavy chain of human gamma 1 immunoglobulins shown in FIG. 3 comprising an insertion of an additional cysteine and of a stop codon (SEQ ID NO: 10);
the recombinant constant region of heavy chain of human gamma 1 immunoglobulins shown in FIG. 4 (SEQ ID NO: 12); and
the recombinant constant region of heavy chain of human gamma 4 immunoglobulins shown in FIG. 5 (SEQ ID NO: 14);

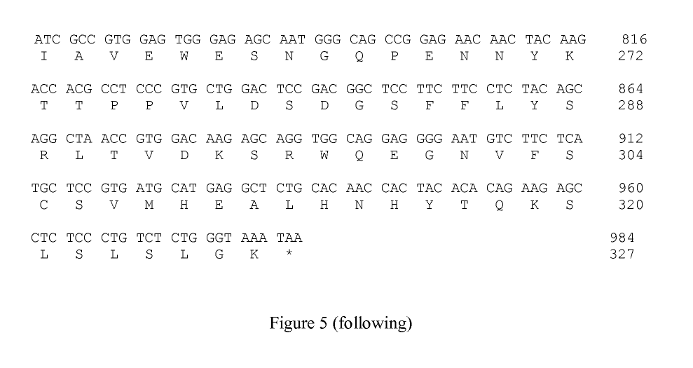

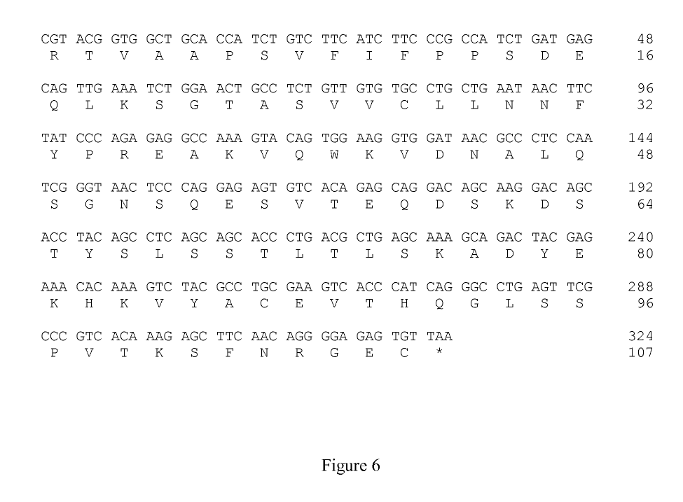

Again advantageously, said antibody comprises the constant region from a human light chain of human kappa immunoglobulins shown in FIG. 6 (SEQ ID NO: 16).

Nevertheless, murine antibodies and sometimes chimeric antibodies are recognized as foreign by a human host and elicit the so-called “human anti-mouse antibody” or “HAMA”response.

The term “humanized antibody” relates to an antibody wherein one or more amino acids of the mouse variable sequence have been substituted by the corresponding one or more amino acids of a homolog human variable sequence in order to reduce the immunogenicity of said mouse variable sequence after its administration to a human subject. This humanization of the variable region of the antibody and eventually the CDR is made by techniques that are by now well known in the art.

A number of methods for producing such humanized antibodies have yet been reported, thus forming part of the general knowledge of the skilled artisan.

As an example, British Patent Application GB 2188638A and U.S. Pat. No. 5,585,089 disclose processes wherein recombinant antibodies are produced where the only portion of the antibody that is substituted is the complementarity determining region, or “CDR”. The CDR grafting technique has been used to generate antibodies which consist of murine CDRs, and human variable region framework and constant regions (See. e. g., RIECHMANN et al., Nature, vol. 332, p: 323-327, 1988). These antibodies retain the human constant regions that are necessary for Fc dependent effector function, but are much less likely to evoke a HAMA response.

As another example, the framework regions of the variable regions are substituted by the corresponding human framework regions leaving the non-human CDR substantially intact, or even replacing the CDR with sequences derived from a human genome (See e.g. Patent application US 2006/258852). Fully human antibodies are produced in genetically modified mice whose immune systems have been altered to correspond to human immune systems. As mentioned above, it is sufficient for use in the methods of the invention, to employ an immunologically specific fragment of the antibody, including fragments representing single chain forms.

A humanized antibody again refers to an antibody comprising a human framework, at least one CDR from a non-human antibody, and in which any constant region present is substantially identical to a human immunoglobulin constant region, i. e., at least about 85 or 90%, preferably at least 95% identical. Hence, all parts of a humanized antibody, except possibly the CDRs, are substantially identical to corresponding parts of one or more native human immunoglobulin sequences. For example, a humanized immunoglobulin would typically not encompass a chimeric mouse variable region/human constant region antibody.

Humanized antibodies have at least three potential advantages over non-human and chimeric antibodies for use in human therapy : 1) Because the effector portion is human, it may interact better with the other parts of the human immune system (e.g., destroy the target cells more efficiently by complement-dependent cytotoxicity (CDC) or antibody-dependent cellular cytotoxicity (ADCC)).

2) The human immune system should not recognize the framework or C region of the humanized antibody as foreign, and therefore the antibody response against such an injected antibody should be less than against a totally foreign non-human antibody or a partially foreign chimeric antibody.

3) Injected non-human antibodies have been reported to have a half-life in the human circulation much shorter than the half-life of human antibodies. Injected humanized antibodies will have a half-life essentially identical to naturally occurring human antibodies, allowing smaller and less frequent doses to be given.

As an example, the design of humanized immunoglobulins may be carried out as follows: When an amino acid falls under the following category, the framework amino acid of a human immunoglobulin to be used (acceptor immunoglobulin) is replaced by a framework amino acid from a CDR-providing non-human immunoglobulin (donor immunoglobulin): (a) the amino acid in the human framework region of the acceptor immunoglobulin is unusual for human immunoglobulin at that position, whereas the corresponding amino acid in the donor immunoglobulin is typical for human immunoglobulin at that position; (b) the position of the amino acid is immediately adjacent to one of the CDRs; or (c) any side chain atom of a framework amino acid is within about 5-6angstroms (center-to-center) of any atom of a CDR amino acid in a three dimensional immunoglobulin model (QUEEN et al., Proc. Natl. Acad. Sci. USA, vol. 88, p:2869, 1991). When each of the amino acid in the human framework region of the acceptor immunoglobulin and a corresponding amino acid in the donor immunoglobulin is unusual for human immunoglobulin at that position, such an amino acid is replaced by an amino acid typical for human immunoglobulin at that position.

In another preferred embodiment, said antibody is a humanized antibody and the light and heavy chain framework sequences are from humanized immunoglobulin light and heavy chains respectively.

A preferred light chain variable region (LCVR) of a humanized antibody of the present invention has an amino acid sequence which differs from at least one amino acid with SEQ ID NO:8. More preferably, the light chain variable region (LCVR) of a humanized antibody of the present invention has the amino acid sequence SEQ ID NO: 25, said amino acid sequence SEQ ID NO: 25 differing from at least one amino acid with SEQ ID NO:8.

This humanized sequence corresponds to the anti-CD 160 murine variable light chain in which one or more amino acid substitutions has been introduced in the framework after comparison with human immunoglobulin kappa variable light chain (SEQ ID NO: 22., Accession number AAZ09098) so as to reduce potential immunogenicity.

A preferred heavy chain variable region (HCVR) of a humanized antibody of the present invention has an amino acid sequence which differs from at least one amino acid with SEQ ID NO:6. More preferably, a heavy chain variable region (HCVR) of a humanized antibody of the present invention has the amino acid sequence SEQ ID NO:26, said amino acid sequence SEQ ID NO:26 differing from at least one amino acid with SEQ ID NO:6.

This humanized sequence corresponds to the anti-CD160 murine variable heavy chain in which one or more amino acid substitutions has been introduced in the framework after comparison with human immunoglobulin variable heavy chains SEQ ID NO: 23 (Accession number 3FCTB) and SEQ ID NO: 24 (Accession number AAG00910) so as to reduce potential immunogenicity.

Preferably, said humanized antibody further comprises the constant regions from human light and heavy chains. As an example, said humanized antibody further comprises the constant regions from human light and heavy chains as described previously.

Other sequences are possible for the light and heavy chains for the humanized antibodies of the present invention. The immunoglobulins can have two pairs of light chain/heavy chain complexes, at least one chain comprising one or more mouse complementarity determining regions functionally joined to human framework region segments.

According to a second aspect, the present invention is related to a pharmaceutical composition comprising at least one anti-CD160 antibody or fragment thereof as described previously and a pharmaceutically acceptable carrier for use in diagnosis and/or prognosis and/or therapy.

Said composition is particularly useful for inhibiting angiogenesis, for the treatment of angiogenic pathologies, and preferably for inhibiting tumor progression.

Said composition may be in any pharmaceutical form suitable for administration to a patient, including but not limited to solutions, suspensions, lyophilized powders, capsule and tablets.

The pharmaceutical compositions of the invention may further comprise any pharmaceutically acceptable diluent, excipient or auxiliary.

The pharmaceutical composition of the invention may be formulated for injection, e.g. local injection, transmucosal administration, inhalation, oral administration and more generally any formulation that the skilled person finds appropriate to achieve the desired prognosis and/or diagnosis and/or therapy.

The anti-CD160 antibody of the invention is contained in said pharmaceutical composition in an amount effective to achieve the intended purpose, and in dosages suitable for the chosen route of administration.

More specifically, a therapeutically effective dose means an amount of a compound effective to prevent, alleviate or ameliorate symptoms of the disease or condition of the subject being treated, or to arrest said disease or condition.

Depending on the intended application, the anti-CD 160 antibody of the invention may further comprise additional constituents.

For example, when the anti-CD160 antibody of the invention is intended for prognosis or diagnosis, it may further comprise a detectable label, such as a fluorochrome, or an entity with enzymatic activity, or with radioactivity, and more generally any entity enabling the detection of said compound. The anti-CD 160 antibody of the invention may of course alternatively be used for the detection of anti-angiogenic sites.

The present invention hence also relates to a pharmaceutical composition or kit comprising at least one anti-CD 160 antibody of the invention, which is intended for the detection of anti-angiogenic sites.

When the anti-CD160 antibody of the invention is intended for therapeutic administration to an organism in need thereof, it may further comprise an immunotoxin and/or a radioelement.

A third aspect of the present invention concerns a method for inhibiting angiogenesis comprising providing to a patient in need thereof a pharmaceutical composition as described herein, which comprises at least one anti-CD160 antibody or fragment thereof as described previously.

As used herein, the term “patient” refers to a mammal, preferably to a human.

Preferably, a patient in need thereof corresponds to a patient suffering from a pathology associated with endothelial cells proliferation engaged in an angiogenesis process.

As an example of such pathologies, one can cites cancer (e.g. tumor vascularization), retinopathies (e.g., diabetic retinopathy), rheumatoid arthritis, chronic graft rejection such as cornea or kidney, acute graft rejection observed in xenograft, angioma, angiosarcoma such as Kaposi's sarcoma or Castelman syndrome, atherosclerosis, endometriosis associated with neovascularization, or tissue overproduction due to cicatrisation.

A forth aspect of the present invention concerns the use of at least one anti-CD 160 antibody or fragment thereof as described previously for the preparation of a medicament for treating and/or preventing a pathology associated with endothelial cells proliferation engaged in an angiogenesis process.

Such a pathology is selected in the group comprising cancer (e.g. tumor vascularization), retinopathies (e.g., diabetic retinopathy), rheumatoid arthritis, chronic graft rejection such as cornea or kidney, acute graft rejection observed in xenograft, angioma, angiosarcoma such as Kaposi's sarcoma or Castelman syndrome, atherosclerosis, endometriosis associated with neovascularization, or tissue overproduction due to cicatrisation.

Other embodiments and advantages of the present invention are illustrated in the following non-limiting examples.

### EXAMPLES

1) Production and Purification of mAb:

MAbs are obtained by immunizing BALB/C mice with a fusion protein between GST and CD-160 (SEQ ID NO:27.; Accession number CAG46665). Cell fusions are carried out with NS1 as described in GOUTTEFANGEAS et al. (Eur. J. Immunol., vol. 22, p:2681-5, 1992).

Screening is performed in two stages. Following indirect immunofluorescence staining and flow cytometry analysis, all hybridoma supernatants reacting with CD 160-GST fusion protein are retained. The cultures containing the selected mAb are cloned twice by limited dilutions.

2) Screening for an Angiogenesis Inhibition Activity:

Growth reduced Matrigel (BD BIOSCIENCES) is diluted in collagen (1/6 v/v) and kept on ice. 160 μl of this solution is added to each well of 8-well culture slides precoated with type I rat tail collagen and left at 37° C. for lh. Following gel formation, a HUVEC suspension, mixed or not with control, FGF-2, sHLA-G1 or hybridoma supernatants is seeded on Matrigel/collagen gels for 24 h at 37° C. in a humidified 5% CO2 incubator.

Angiogenesis is quantified as described in RUGGERI et al. (Cancer Res., vol. 63(18), p:5978-91, 2003).

Briefly, the culture medium is removed, the cells are rinsed twice with PBS and fixed for 30 min at room temperature in a 4% PFA solution. Then, the cells are washed twice with PBS and stained with Masson's Trichrom stain. The extent of microcapillary network is measured using an automated computer-assisted image analysis system (Imagenia, Biocom, Les Ulis, France), and the total length of the capillaries in each well is determined. The mean microcapillary network length (μm) is calculated for each experimental condition. Experiments are performed in triplicate and repeated 3 times.

Suprisingly, one supernatant among all the hybridoma's supernatants leads to the inhibition of FGF-2 mediated tubule vessel growth demonstrating that the antibodies of this specific supernatant are able to inhibit angiogenesis.

3) Cloning of the Variable Regions of the Anti-CD160 Antibody having Anti-Angiogenesis Properties:

The specific hybridoma producing antibodies inhibiting angiogenesis is isolated. Total RNA was extracted from cells of said hybridoma with RNeasy kit (QIAGEN) according to the instruction of the manufacturer.

50 μg of total RNA as described in CHARDES et al. (FEBS letters, vol. 452, p:386-94, 1999) with primers specific of constant regions from the kappa light chain and from the gamma variable chain respectively. Then, the protocol described in CHARDES et al. (1999, abovementioned) was used for screening and isolating the variable regions of the immunoglobulin of interest.

This screening has enable to identify the variable regions of the light chain and of the heavy chain of the immunoglobulin of interest (FIGS. 1 and 2).

These sequences were analysed by an alignment with other immunoglobulins on the IMGT/V-QUEST site (http://imgt.cines.fr/IMGT_vquest/share/textes/index.html) for identifying the framework region (FR) and the CDR position.

The CDR position as identified for the variable heavy and light chains are presented in FIGS. 1 and 2 respectively.

4) Humanization of the Variable Regions of the Anti-CD 160 Antibody having Anti-Angiogenesis Properties:

For the immunization of the identified variable regions are aligned with the human immunoglobulins having the maximum of homology with said variables regions.

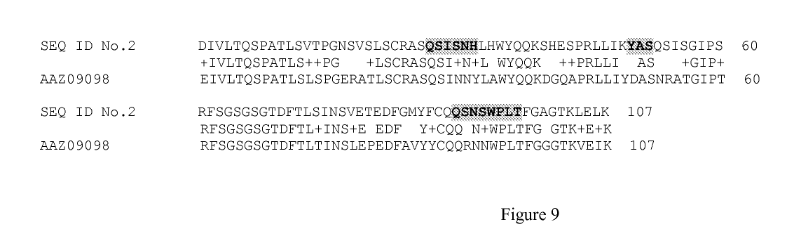

Human immunoglobulin having the maximum of homology (SEQ ID NO: 22; accession number AAZ09098) with the mouse variable light chain SEQ ID NO: 8. is shown in FIG. 9.

In view of this alignment, multiple substitutions are tested in order to humanize the mouse variable light chain. The tested substitutions are as follows:

position 17 (of SEQ ID No.8): N→E;

position 18: S→R

position 19: V→A

position 41: H→G

position 53 : Q→N

position 80: T→P

position 84 : G→A

position 100: A→G

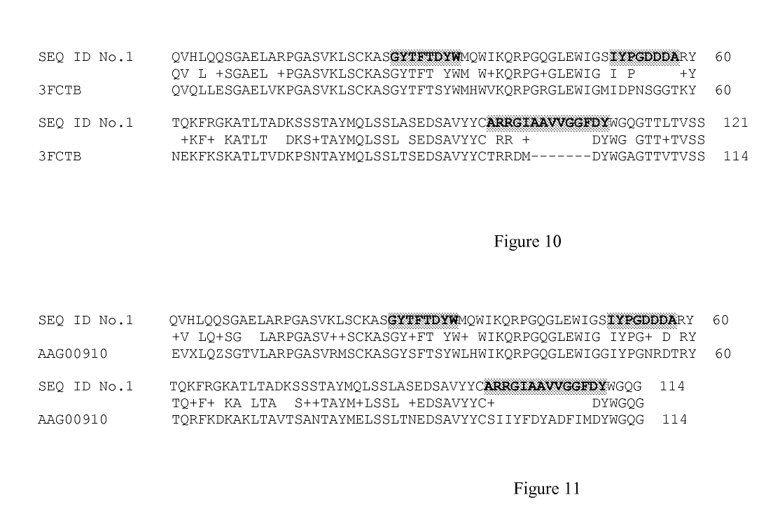

Human immunoglobulins having the maximum of homology (SEQ ID NO: 23 and SEQ ID NO: 24; accession number 3FCTB and AAG00910 respectively) with the mouse variable heavy chain SEQ ID No.1 are shown in FIGS. 10 and 11 respectively.

In view of this alignment, multiple substitutions are tested in order to humanize the mouse variable heavy chain. The tested substitutions are as follows:

position 3 (of SEQ ID No.6): H→Q;

position 35: Q→H

position 87: A→T

5) Construction of Chimeric Antibodies:

The sequences of the variable heavy and light chains, humanized or not, are combined with signal peptides and their respective human kappa and IgG1 or IgG4 constant region domains such as described in LEUNG et al. (Hybridoma, vol. 13(6), p:469-76, 1994).

The anti-angiogenic activity of these antibodies is tested as described previously (cf. 2).

1. An antibody or fragment thereof, capable of binding to CD160, said antibody having a polypeptidic sequence comprising at least 90% homology with the polypeptidic sequence of an antibody comprising:
a) a light chain comprising three light chain complementary regions (CDRs) having the following amino acid sequences:
i) the light chain CDR1: QSISNH (SEQ ID NO:1);
ii) the light chain CDR2: YAS;
iii) the light chain CDR3: QQSNSWPLT (SEQ ID NO: 2); and
a light chain framework sequence from an immunoglobulin light chain; and
b) a heavy chain comprising three heavy chain complementary regions (CDRs) having the following amino acid sequences:
i) the heavy chain CDR1: GYTFTDYW (SEQ ID NO: 3);
ii) the heavy chain CDR2: IYPGDDDA (SEQ ID NO: 4);
iii) the heavy chain CDR3: ARRGIAAVVGGFDY (SEQ ID NO: 5); and
a heavy chain framework sequence from an immunoglobulin heavy chain.

2. The antibody of claim 1, comprising:
a) a light chain comprising three light chain complementary regions (CDRs) having amino acid sequences having at least 90% homology with the following amino acid sequences i) to iii):
i) the light chain CDR1: QSISNH (SEQ ID NO:1);
ii) the light chain CDR2: YAS;
iii) the light chain CDR3: QQSNSWPLT (SEQ ID NO: 2); and
a light chain framework sequence from an immunoglobulin light chain; and
b) a heavy chain comprising three heavy chain complementary regions (CDRs) having amino acid sequences having at least 90% homology with the following amino acid sequences i) to iii):
i) the heavy chain CDR1: GYTFTDYW (SEQ ID NO: 3);
ii) the heavy chain CDR2: IYPGDDDA (SEQ ID NO: 4);
iii) the heavy chain CDR3: ARRGIAAVVGGFDY (SEQ ID NO: 5); and
a heavy chain framework sequence from an immunoglobulin heavy chain.

3. The antibody of claim 1 or 2, comprising:
a) a light chain comprising three light chain complementary regions (CDRs) having the following amino acid sequences:
i) the light chain CDR1: QSISNH (SEQ ID NO:1);
ii) the light chain CDR2: YAS;
iii) the light chain CDR3: QQSNSWPLT (SEQ ID NO: 2); and
a light chain framework sequence from an immunoglobulin light chain; and
b) a heavy chain comprising three heavy chain complementary regions (CDRs) having the following amino acid sequences:
i) the heavy chain CDR1: GYTFTDYW (SEQ ID NO: 3);
ii) the heavy chain CDR2: IYPGDDDA (SEQ ID NO: 4);
iii) the heavy chain CDR3: ARRGIAAVVGGFDY (SEQ ID NO: 5); and
a heavy chain framework sequence from an immunoglobulin heavy chain.

4. The antibody of any one of claims 1 to 3, wherein said antibody or fragment thereof is capable of binding to CD160 with a K_{D} of less than 10⁻⁷ M, preferably from less than 10⁻⁸ M.

5. The antibody of any one of claims 1 to 4, wherein said antibody comprises the light chain variable region (LCVR) with the amino acid sequence SEQ ID NO: 8, preferably SEQ ID NO:18 or SEQ ID NO:28.

6. The antibody of any one of claims 1 to 5, wherein said antibody comprises the heavy chain variable region (HCVR) with the amino acid sequence SEQ ID NO:6, preferably SEQ ID NO:20.

7. The antibody of any one of claims 1 to 4, wherein said antibody comprises a light chain variable region (LCVR) which amino acid sequence differs from at least one amino acid with SEQ ID NO:8.

8. The antibody of claim 7, wherein said antibody comprises a light chain variable region (LCVR) having the amino acid sequence SEQ ID NO: 25.

9. The antibody of any one of claim 1 to 4 or 8, wherein said antibody comprises a heavy chain variable region (HCVR) which amino acid sequence differs from at least one amino acid with SEQ ID NO:6.

10. The antibody of claim 9, wherein said antibody comprises a heavy chain variable region (HCVR) having the amino acid sequence SEQ ID NO:26.

11. The antibody of any one of claims 1 to 10, wherein said antibody further comprises the constant regions from human light and heavy chains.

12. The antibody of claim 11, wherein said antibody comprises:
a) a constant region from a human heavy chain selected in the group comprising:
the recombinant constant region of heavy chain of human gamma 1 immunoglobulins having the sequence SEQ ID NO: 10, which sequence comprises an insertion of an additional cysteine and of a stop codon;
the recombinant constant region of heavy chain of human gamma 1 immunoglobulins having the sequence SEQ ID NO: 12; and
the recombinant constant region of heavy chain of human gamma 4 immunoglobulins having the sequence SEQ ID NO: 14; and
b) the constant region from human light chain of human kappa immunoglobulins having the sequence SEQ ID NO: 16.

13. The antibody of claim 12, wherein said antibody comprises the recombinant constant region of heavy chain of human gamma 1 immunoglobulins having the sequence SEQ ID NO: 10 and the constant region from human light chain of human kappa immunoglobulins having the sequence SEQ ID NO: 16.

14. A pharmaceutical composition comprising at least one antibody according to any one of claims 1 to 13, and a pharmaceutically acceptable carrier.

15. Use of at least one antibody as defined in any of claims 1 to 13, for the preparation of a medicament for treating and/or preventing a pathology associated with endothelial cells proliferation engaged in an angiogenesis process.

16. The use of claim 15, wherein said pathology is selected in the group comprising cancer, retinopathies, rheumatoid arthritis, chronic graft rejection, acute graft rejection observed in xenograft, angioma, angiosarcoma, atherosclerosis, endometriosis associated with neovascularization, and tissue overproduction due to cicatrisation.

17. The use of claim 16, wherein said pathology is selected in the retinopathies.

18. The use of claim 17, wherein the antibody is as defined in claim 13.
